(19) United States
(12) Patent Application Publication
(10) Pub. No.: US 2024/0043906 A1
(43) Pub. Date: Feb. 8, 2024

(54) METHOD OF ELUTING NUCLEIC ACID

(71) Applicant: UNIVERSITY OF DUNDEE, Dundee (GB)

(72) Inventor: Alexander GRAY, Grampian (GB)

(21) Appl. No.: 18/268,028

(22) PCT Filed: Dec. 23, 2021
(86) PCT No.: PCT/GB2021/053422

§ 371 Date: Jun. 16, 2023

(30) Foreign Application Priority Data
Dec. 24, 2020 (GB) 2020576.1

(51) Int. Cl. C12Q 1/6806; C12N 15/10

(52) U.S. Cl. CPC C12Q 1/6806; C12N 15/1003

(57) ABSTRACT

The invention relates to a method of eluting nucleic acid from a swab. The invention also relates to a kit for eluting nucleic acid from a swab and an elution solution.

### FIELD OF THE INVENTION

The present invention relates to a method of eluting nucleic acid from swabs containing cellulose. The invention also relates to a kit containing the elution solution and the elution solution.

### BACKGROUND

DNA recovery from swabs is performed in many different areas of science. For example, in forensic medicine where a high percentage of recovery is needed as the concentration of nucleic acid on the swab may be minimal. The more that is recovered from the swab, the better the PCR analyses to identify the nucleic acid.

Current methods of eluting DNA from swabs do not have high rates of recovery.

### SUMMARY OF THE INVENTION

The inventors have developed a method which can recover 70% or more nucleic acid from swabs comprising cellulose.

In a first aspect, there is provided a method of eluting nucleic acid from a swab comprising cellulose, the method comprising incubating the swab in an elution solution, the elution solution comprising:
a) A polymer comprising pyrrolidone side chains;
b) A non-ionic or zwitterionic detergent;
c) A protease; and
d) A buffer at pH 5-8.5.

In a further aspect, the invention provides a kit for eluting nucleic acid from a swab comprising cellulose, the kit comprising:
a) A polymer comprising pyrrolidone side chains, preferably PVP (polyvinylpyrrolidone); and any one or more of the following:
b) A non-ionic or zwitterionic detergent, preferably polysorbate 20 (e.g. Tween® 20); and/or
c) A protease, preferably proteinase K; and/or
d) A buffer at pH 5-8.5; and/or
e) One or more spin baskets.

In a further aspect, the invention provides an elution solution comprising:
a) A polymer comprising pyrrolidone side chains; and
b) A non-ionic or zwitterionic detergent, preferably polysorbate 20; and optionally c) A buffer at pH 5-8.5.

### DETAILED DESCRIPTION

Eluting Nucleic Acid

By eluting nucleic acid is meant disrupting the nucleic acid binding to the swab to extract the nucleic acid away from the swab. The elution solution contains compounds which can disrupt this binding.

Nucleic Acid

The nucleic acid to be eluted may be any which is of interest to analyse which is interacting with the cellulose in the swab. For example, animal or human RNA/DNA or RNA or DNA from a microorganism, for example a bacteria or virus.

The DNA/RNA may be cell-free DNA/RNA and/or cellular DNA/RNA. The cellular DNA/RNA is released from cells when the swab is incubated in the elution solution.

Swab Comprising Cellulose

A swab in forensics is a small tool used to collect a forensic sample, for example, a sample containing animal or human DNA. Collection is typically done via the head of the tool which is made of a material suitable for collecting a forensic sample.

Swabs comprising cellulose, e.g. cotton swabs, are superior than swabs made of other materials (e.g. nylon or rayon) at collecting DNA. This is because the cellulose binds the DNA. However, although cotton swabs are superior in collecting DNA, it is difficult then to remove the DNA from the swab for analyses.

The elution solution described breaks up the interactions between the cellulose and DNA or RNA. The swab can be any which contains cellulose. For example a cotton swab.

Swab may be substituted for any other cellulose-based nucleic acid collector. That is, the method elutes nucleic acid from any cellulose-based material which can be used to collect nucleic acid.

Elution Solution

The elution solution removes the nucleic acid from the cellulose allowing effective recovery of the nucleic acid for analyses. Importantly, the elution solution also does not interfere with subsequent PCR meaning the nucleic acid does not have to be recovered from the elution solution before PCR can be performed. PCR or other sequencing reaction can be performed on the elution solution without isolating the nucleic acid from the elution solution.

The amount of elution solution required to incubate the swab can be titrated by the person skilled in the art. The whole surface of the swab must be immersed to maximise DNA recover. However, the minimal volume may be 250 μl.

For example, when using a swab head of 1 cm and 0.5 cm in diameter, 250 μl of elution solution may be used.

Polymer

The polymer used in the elution solution disrupts the DNA binding to the cellulose. The polymer is a synthetic polymer.

The polymer comprises pyrrolidone side chains. That is, the pyrrolidone is a pendant group. The pyrrolidone may be 2-pyrrolidone which has the following structure:

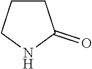

The pyrrolidone ring may be further substituted at any position on the ring. For example, the ring may be substituted at carbons 3, 4 and/or 5 of the ring, with any one or more of the following substituents: alkyl groups, for example C1, C2, C3 or C4 alkyl groups; halogens; hydroxyl groups and/or acid groups.

Alternatively, the pyrrolidone ring may not be substituted.

The polymer comprises at least 2 pyrrolidone side chains, preferably the polymer comprises 100 or more pyrrolidone side chains.

The polymer backbone may comprise or consist of carbon atoms. The polymer may be a homopolymer or a copolymer.

The polymer may comprise vinyl pyrrolidone monomers, e.g. 1-vinyl-2-pyrrolidinone monomers. The polymer may be a copolymer comprising these monomers as well as other monomers.

The linkage between the backbone and the side chains in the polymer may be via the nitrogen atom.

The polymer comprising pyrrolidone side chains may be PVP (polyvinylpyrrolidone).

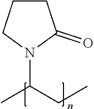

where n can be any number.

PVP (Polyvinylpyrrolidone)

PVP, also commonly called polyvidone or povidone, is a polymer made from the monomer N-vinylpyrrolidone.

The concentration of PVP may be 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4 or 5% w/v.

Any PVP may be used.

The PVP may be PVP40 where the polymers have an average molecular weight of 40 KDa. Alternatively, the PVP may be PVP10 where the polymers have an average molecular weight of 10 KDa.

The PVP may have an average molecular weight of 45 kDa or below, for example, 40, 35, 30, 25, 20, 15 or 10 kDa or any range within these molecular weights. For example, from about 45 kDa to about 10 kDa, or from about 45 KDa to about 20 KDa, or from about 20 KDa to about 10 KDa, or from about 15 kDa to about 25 kDa.

Detergent

The detergent in the elution buffer is a non-ionic or a zwitterionic detergent.

The detergent may be in a concentration of 0.2, 0.4, 0.5, 1, 2, 3, 4 or 5%.

The detergent may be any non-ionic surfactant derived from sorbitan esters. For example, any polysorbate. For example, polysorbate 20 (polyoxyethylene (20) sorbitan monolaurate). Tweens® are a series of non-ionic surfactants derived from sorbitan esters. Tween® 20 may be used.

Other detergents may also be used, for example Zwittergent® 3-08 (n-Octyl-N,N-dimethyl-3-ammonio-1-propanesulfonate).

Examples of Elution Solutions

The elution solution may comprise a polymer comprising pyrrolidone side chains, for example PVP, at a concentration of 1-2%. The elution solution may comprise detergent at a concentration of 0.5-1.5%.

The protease added to these concentrations of PVP and detergent may be 50-150 μg/ml, for example 100 μg/ml.

Percentages

The percentages of the individual components described are % w/v. That is, weight per volume.

For example, a 1 ml solution with 1% Tween® 20 and 1% PVP40 may be made up as follows:
100 μl of 10% (w/v) Tween® 20 (stock Tween® solution is difficult to pipette due to viscosity);
100 μl of 10% (w/v) PVP40 (stock PVP is a powder);
100 μl of 200 mM stock solution buffer, for example TrisHCl to make 20 mM TrisHCl in the final 1 ml solution;
5 μl of 20 mg/ml protease, for example Proteinase K to make 0.1 mg/ml in the final 1 ml solution; and
695 μl water.

Protease

The function of the protease is to remove contaminating proteins from the cellular DNA, e.g. histone proteins, and to destroy nucleases which may be released when the cell is ruptured.

The protease may be present in the eluting solution at a concentration of at least 40 μg/ml. For example, at a range of 40-400 μg/ml. For example, 40-150 μg/ml in the elution solution. For example, 100 μg/ml in the elution solution.

The protease may have activity at a temperature of between 50-65° C., for example 50-60° C. The protease may be active in the presence of detergents. The protease may have more than 1 site of cleavage.

The protease may be proteinase K. Other broad-spectrum proteases which can digest histone proteins and release cellular DNA would be known to the skilled person. For example, Nagarse (subtilisin BPN) and other subtilisin derived proteases.

Buffer

The buffer may be any which maintains the pH at 5-8.5. For example, the buffer may be Tris HCl. Preferably the buffer maintains the pH at pH 7-8.

The concentration of the buffer may be 5 mM, 10 mM, 15 mM, 20 mM, 25 mM, 30 mM, 40 mM or 50 mM. For example, the buffer may be at a concentration of at least 5 mM up to 150 mM to allow buffering of the solution.

Kit

The kit comprises:
a) A polymer comprising pyrrolidone side chains, preferably PVP; and any one or more of the following:
b) A non-ionic or zwitterionic detergent, preferably polysorbate 20; and/or
c) A protease, preferably proteinase K; and/or
d) A buffer at pH 5-8.5; and/or
e) One or more spin baskets.

In the kit, a minimal elution solution may comprise:
a) A polymer comprising pyrrolidone side chains; and
b) A non-ionic or zwitterionic detergent;
and optionally
c) A buffer at pH 5-8.5.

The protease may be separate from the above minimal solution in the kit and is added before addition of the elution solution to the swab.

The spin baskets can be used to remove the swab from the elution solution.

The individual components of the kit are as per the descriptions above.

Incubating the Substrate

The incubation comprises immersing the swab into the elution solution. The swab may be completely covered by elution solution.

The incubation may be carried out at 40-60° C. For example, 50-60° C. For example, 56° C. The incubation may be carried out at this temperature using a water bath, dry bath or block heater.

The incubation can be carried out for at least 10 mins, 20 minutes, 30 minutes, or 1 hour or more, for example 1-3 hours.

For example, the incubation can be carried out for at least 1 hour at 50-60° C. The incubation may be for at least 1 hour at 52, 53, 54, 55, 56, 57, 58, 59 or 60° C.

Centrifuging the Swab

To collect the elution solution containing the eluted DNA after incubation, the swab may be centrifuged. This ensures all of the elution solution is collected from the swab. Therefore, the maximum amount of DNA is recovered.

Centrifugation may be carried out by inserting the swab in a tube with a small hole in the bottom. This tube may then be inserted into an Eppendorf tube. Upon centrifugation, the elution solution will spin down into the Eppendorf and the swab will remain in the tube with the small hole.

Otherwise, a spin basket may be used. These are also known as spin filter baskets. These are small baskets with holes in the bottom which can be inserted into an Eppendorf. The holes may be of a size of around 50 microns. The swab will be remain in the spin basket. The elution solution will be collected into the Eppendorf upon centrifugation.

Heating the Collected Elution Solution

After incubation and centrifugation, the collected elution solution containing the eluted DNA may be heated to at least 90° C., preferably at least 95° C. This ensures the protease activity is destroyed prior to PCR.

Alternatively, the collected elution solution is PCRed without heating. Protease activity can be destroyed by the PCR cycle.

Washing with Ethanol

Before incubation, the swab may be washed with ethanol. This removes oil or salt on the swab which may affect PCR. The swab may be washed with 70-80% ethanol before incubation in the elution solution.

The wash volume will depend on the size of the swab. For example, for a swab of size 1 cm by 0.5 cm diameter, a wash volume of 0.3-0.5 ml 70-80% ethanol may be used. The swab may be washed and then centrifuged to remove the ethanol. For example, by inserting the swab in a spin-basket. The DNA does not elute with the ethanol and remains bound to the cellulose.

PCR

The eluted DNA collected from after discarding the spin basket can be used directly in PCR. That is, the DNA can be directly PCRed from the elution solution. The elution solution does not interfere with downstream PCR. Therefore, after incubation of the swab, the eluted DNA can be used directly in PCR.

Alternatively, the DNA can be concentrated before PCR or other analyses. Concentrating the DNA may be carried out using magnetic beads for example.

Throughout the specification, unless the context demands otherwise, the terms ‘comprise’ or ‘include’, or variations such as ‘comprises’ or ‘comprising’, ‘includes’ or ‘including’ will be understood to imply the method or kit includes a stated integer or group of integers, but not the exclusion of any other integer or group of integers.

Each document, reference, patent application or patent cited in this text is expressly incorporated herein in their entirety by reference, which means it should be read and considered by the reader as part of this text.

The invention is further described below by way of example only, and with reference to the accompanying figures.

### DESCRIPTION OF THE FIGURES

FIG. 1: Comparison of 1-2% PVP in extraction of free DNA dried onto cotton swabs.

FIG. 2: Proteinase K treatment releases the cellular DNA from swabs. 1e6 cells or 93 ng free DNA was dried onto swabs and then extracted in the presence or absence of proteinase K followed by heat killing the proteinase K.

FIG. 3: Agarose gels of PCR products from cellular material treated as indicated in Example 3.

FIG. 4: Diagram of different ways of collecting DNA on swab, see Example 4.

FIG. 5: 10 ul samples were taken before heat treatment to kill the proteinase K and DNA concentration estimated by Qubit dye binding assay. Panel A (top): shows estimated DNA in ng and Panel B (bottom) shows percentage DNA recovered relative to the direct to buffer controls (DB).

FIG. 6: This figure shows the recovery and % recovery (relative to the direct to buffer controls) of free DNA deposited directly into extraction buffer, onto a cotton swab or recovered by swabbing a glass slide using a cotton swab. DNA content was estimated by qPCR (sybre Green) using trout primer pair 2 (free DNA).

FIG. 7: This figure shows the recovery and % recovery (relative to the direct to buffer controls) of cellular DNA deposited directly into extraction buffer, onto a cotton swab or recovered by swabbing a glass slide using a cotton swab. DNA content was estimated by qPCR (sybre Green) using mouse primer pair 1 (cellular DNA).

FIGS. 8-9: FIGS. 8 and 9 shows that PVP outperforms other polar polymers such as dextran (D). However, other detergents work in the elution solution as well as Tween, here we show Zwittergent 3-08 (Z).

FIGS. 10-11: These figures compare the recovery of DNA using a commercially available Qiagen™ kit and the claimed method.

### EXAMPLES

### Example 1: Comparison of Different Amounts of PVP for Elution from Swabs

Method:
1. 10 ng/ul Trout DNA stock. Spotted 5 ul=50 ng onto swabs and dried.
2. Elution solution: 1% to 2% PVP40, 1% tween 20 in 20 mM TrisHCl·DNA quantified by Qbit.
3. Extracted 30 min on shaker at 56 degrees in 200 ul of buffer.
4. DNA quantified by Qbit.

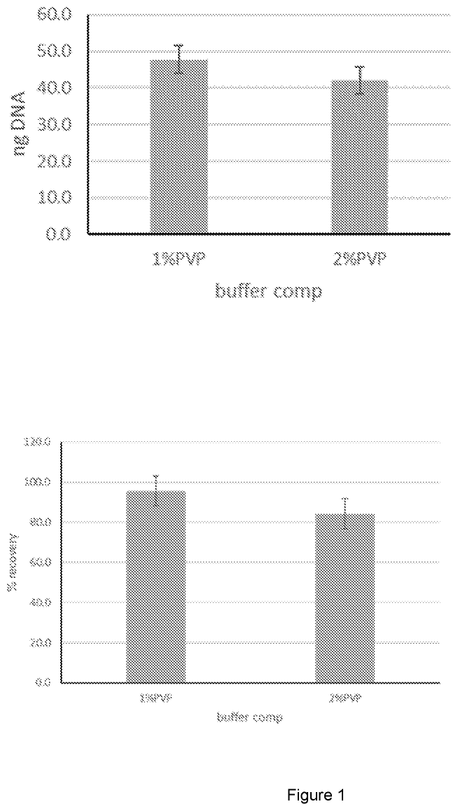

FIG. 1 shows that 1 and 2% PVP have high rates of recovery of DNA from the swab.

### Example 2: The Addition of Proteinase K Enhances Recovery of DNA from Swabs

Method:
1. Swabs were seeded with Stock DNA 10 ul, Qbit verified at 9.3 ng/ul total of 93 ng.
2. Swabs also seeded with PBS washed cells 10 ul of 1×10⁶ cells/ml suspension approximately 60 ng DNA.
3. Controls with DNA or cells as above added directly to 200 ul conditions 1 to 2 as below.
A: EBP (20 mM TrisHCl, pH 7.5; 1% Tween20 and 1% PVP)
B: EBP plus Proteinase K (PK) at 100 μg/ml.
4. Swabs were incubated in elution solution at 56° C. for 1 hour.
5. The swabs were then added to a spin basket inserted into an Eppendorf tube. The spin baskets and Eppendorfs were then spun for 3 minutes at full speed. The spin basket was thrown away.
6. The solution collected in the Eppendorf was heated to 95° C. for 10 minutes to destroy Proteinase K activity.
7. A sample of 10 μl was taken from the Eppendorf (into which the elution solution and eluted DNA were collected) for Qbit analyses.

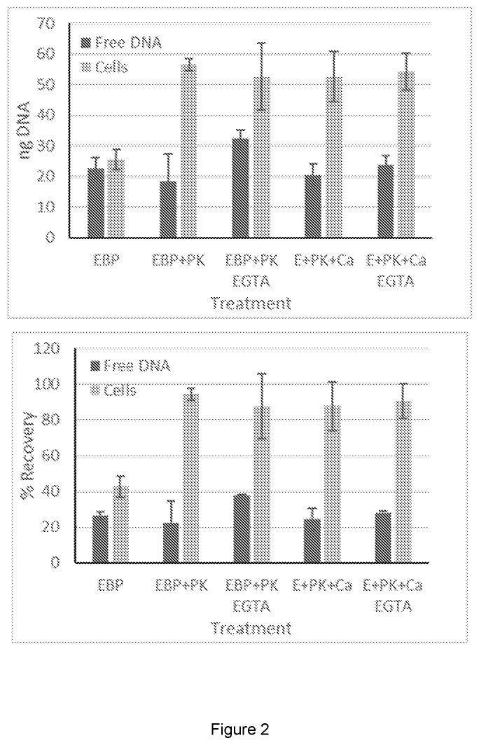

FIG. 2 shows that the proteinase K has a marked effect on the release of cellular DNA. In addition, the presence or absence of calcium or EGTA has no effect

### Example 3: Test of DNA Extraction from Cells in PVP Buffers and Suitability for PCR

Method:
1. Approximately 10\vec{ }6 cells were resuspended in PBS and 5 ul added to 200 ul of the PVP buffers.
2. Samples and buffer blanks were incubated at room temperature for 30 min and EGTA then added where indicated without heat kill at 95 degrees.
3. Samples were also incubated at 56 degrees for 30 min and then EGTA added where indicated. Samples were also incubated at 56 degrees for 30 min and then EGTA added where indicated and heated to 95 degrees for 10 min.
4. 10 ul samples were then taken for PCR annealing temp 68 degrees.

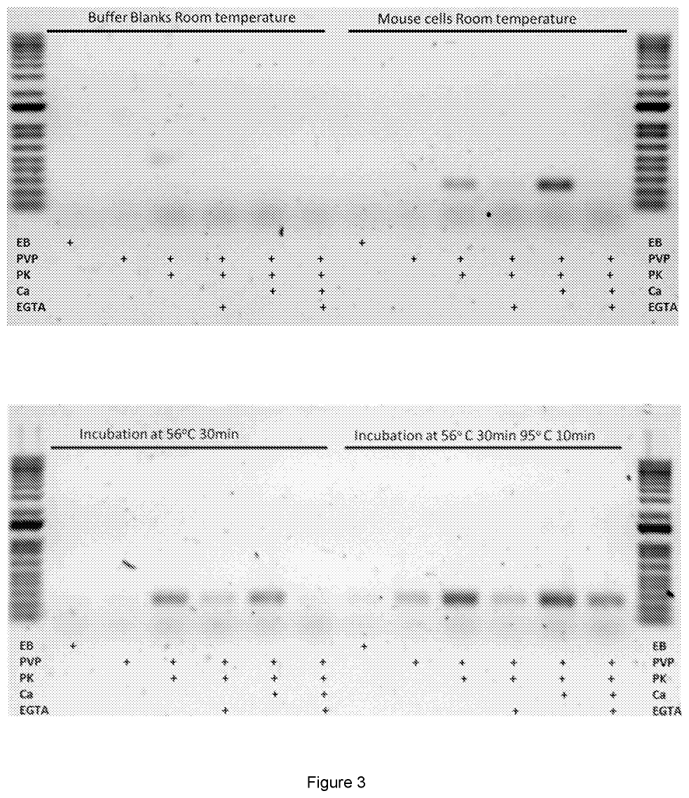

Results are shown in FIG. 3. Incubation at 56 degrees enhances the digestion with EGTA reducing the yield. Including the 95 degree step enhances yield across the board probably due to the proteinase K (PK) being completely destroyed and removing any inhibitory effect on the PCR reaction. There is still PCR in the presence of active Proteinase K. This may be due to the 2 min 95 degree activation step in the PCR cycles which may be enough to destroy the PK activity.

The results of these experiments indicate that the addition of calcium or EGTA is not required for the inactivation of the proteinase K and will be omitted from all future experiments as simple heat treatment seems to be sufficient to inactivate the proteinase K and allow PCR.

### Example 4: Comparing Free and Cellular DNA Recovery from Swab

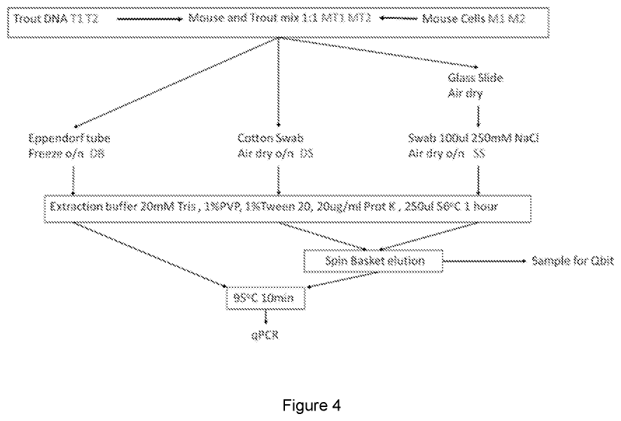

Trial run of a comparison of extraction/recovery efficiency using the elution solution using free trout DNA and cellular mouse DNA. The protocol is show diagrammatically in FIG. 4.

All samples were extracted at 56 degrees for 60 min and then spin basket collected (in the case of swabs)

10 ul samples were taken for Qbit before heating at 95 degrees for 10 min.

Free trout DNA was 14.5 ng/ul and diluted 1:3=4.83 ng/ul seeded at 10 and 5 ul ie: 48.3 ng and 24.1 ng As estimated by Qubit Mouse cells were resuspended in PBS at approx. 0.72e6/ml=4.32 ng/ul and seeded at 10 and 5 ul approximating to 43.2 and 21.6 ng DNA.

Samples with a 1:1 mix of trout cells and trout DNA were also used. Sample ID key is given below:
DB=direct to buffer; Material added directly to 250 ul PVP PK buffer
DS=direct onto swab; Material added directly to a swab and dried over night
SS=slide followed by swab; Material dried onto a glass slide swabbed with cotton swab wetted with 100 ul 250 mM NaCl and dried overnight.
T1 and T2 high and low trout free DNA
M1 and M2 high and low cells.
MT1 and MT2 high and low mixture of free and cellular DNA.

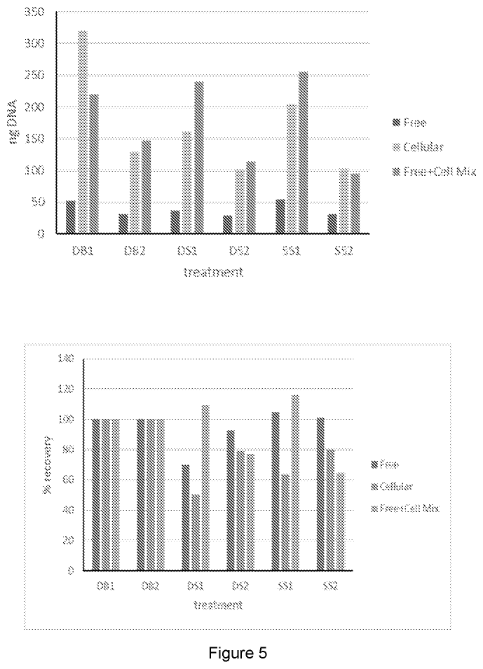

The results of the Qubit assay are shown in FIG. 5.

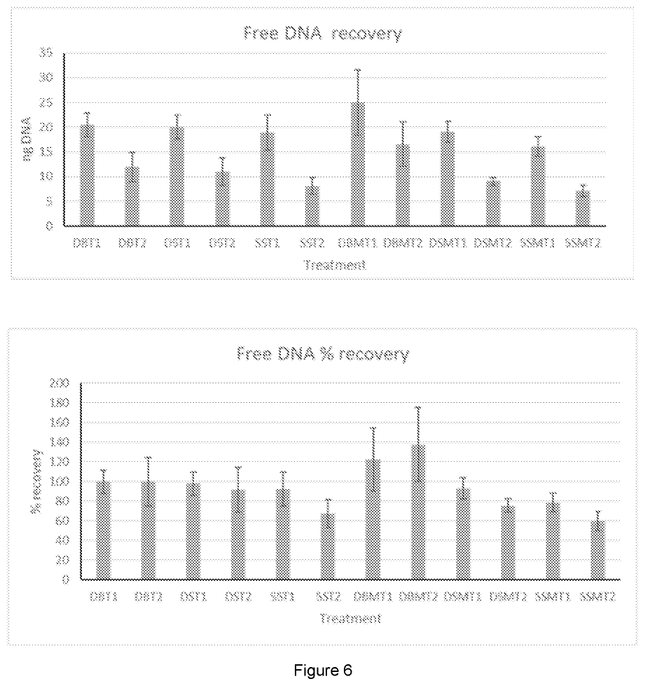

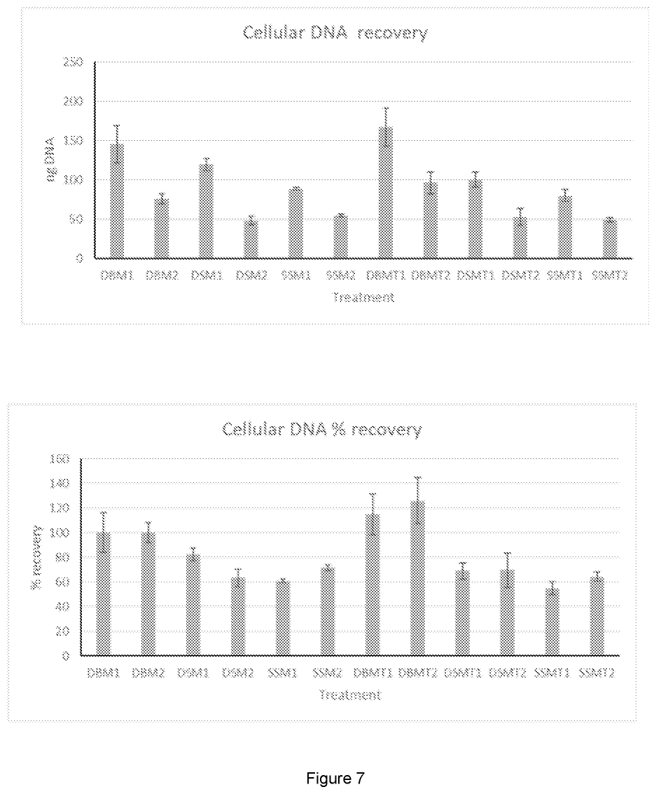

The Qubit data corresponds well with the estimated free DNA but suggests that the input from cellular material is underestimated. As a result, qPCR was ran and the results are shown in FIGS. 6 and 7.

The results relative to the direct to buffer control samples as shown in the data expressed as percent recovery clearly shows 80% to 90% recovery of free DNA from material applied directly to swabs and 70% to 80% of cellular DNA applied directly to swabs. For free DNA the recovery from the swabbed glass slide is still high at 70% to 80% of the applied material and for cellular DNA this is only slightly reduced at around 60% to 70% of the applied material.

### Example 5: Method with Zwittergent 3-08

The effect of substitution with another high molecular weight polymer Dextran Sulphate and substitution of the detergent Tween 20 with Zwittergent 3-08 was investigated.

Swabs were spotted with either 55 or 27.5 ng (10 or 5 ul 5.5 ng/ul) Trout DNA and dried overnight. DNA extraction was carried out with 250 ul of extraction buffers, as detailed below, with shaking at 56° C. for 1 hour. DNA extracted was quantified by Qbit analysis of a 10 ul sample and qPCR of a 5 ul sample.

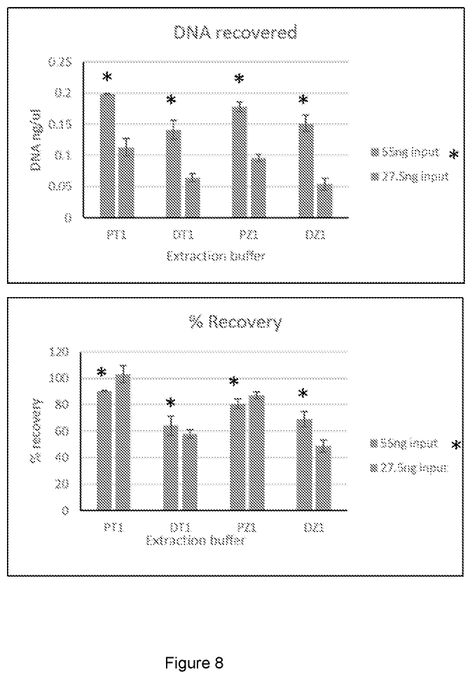

The results of the extraction and Qbit quantification are shown in FIG. 8.

From the data there appears to be a decrease of around 30-40% in the effectiveness of the extraction buffer when PVP is replaced by Dextran sulphate (DT1) and a small decrease in the effectiveness when Tween 20 is replaced by Zwittergent 3-08 (PZ1).

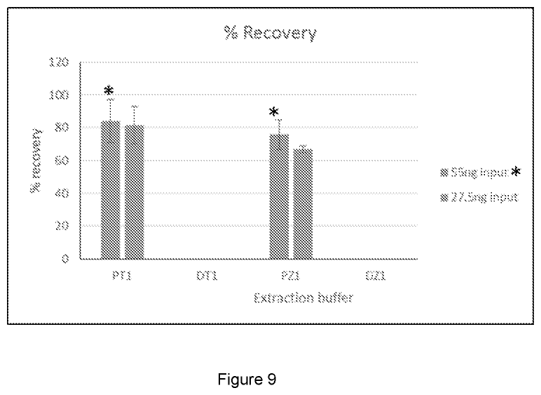

Subsequent quantitation of the DNA by qPCR resulted in the data shown in FIG. 9.

[symbol]

| | | Op [symbol] quant ng/5 ul | | | | Total DNA recovered | | | |
|---|---|---|---|---|---|---|---|---|---|
| [symbol] ng in | PT1 | 0.430 [symbol] 15 | 0.445414 | 0.595155 | 0.431461 | 41.9 [symbol] 1 | 43.36102 | 57.93834 | 42.00276 |
| | DT1 | ND | ND | ND | ND | ND | ND | ND | ND |
| | PZ1 | 0.518175 | 0.633273 | 0.682663 | 0.811742 | 32.69685 | 39.9595 | 43.07603 | 51.2209 [symbol] |
| | DZ1 | ND | ND | ND | ND | ND | ND | ND | ND |
| 27.5 ng in | PT2 | 0.220086 | 0.25 [symbol] 151 | 0.2 [symbol] 51 | 0.18 [symbol] 107 | 21.42533 | 25.1 [symbol] 096 | 24.60288 | 18.31218 |
| | DT2 | ND | ND | ND | ND | ND | ND | ND | ND |
| | PZ2 | 0.2338 [symbol] | 0.3104 [symbol] 9 | 0.314482 | 0.30 [symbol] 786 | 14.75708 | 19.58 [symbol] 7 | 19.84383 | 19.2 [symbol] |
| | DZ2 | ND | ND | ND | ND | ND | ND | ND | ND |

| | | % Recovered | | | | ave | sd |
|---|---|---|---|---|---|---|---|
| [symbol] ng in | PT1 | 76.25419 | 78.83822 | 105.3524 | 76.3 [symbol] 5 | 84.20087 | 13.18181 |
| | DT1 | ND | ND | ND | ND | ND | ND |
| | PZ1 | 59.44882 | 72.65363 | 78.32005 | [symbol] 3.12893 | 75.88786 | 9.05667 |
| | DZ1 | ND | ND | ND | ND | ND | ND |
| 27.5 ng in | PT2 | 77. [symbol] 10 [symbol] | [symbol] 1.38529 | 89.79228 | 66.58975 | 81.41941 | 11.34702 |
| | DT2 | ND | ND | ND | ND | ND | ND |
| | PZ2 | 53.6621 | 71.23627 | 72.15 [symbol] 7 | 70.16395 | 66. [symbol] 0542 | 2.337387 |
| | DZ2 | ND | ND | ND | ND | ND | ND |

[symbol]

indicates data missing or illegible when filed

The data clearly shows that Dextran Sulphate is a potent inhibitor of the PCR reaction since no product was detectable in any samples in which it was present. This makes Dextran sulphate unsuitable as a substitute for PVP (as well as that Dextran does not remove the nucleic acid from the cellulose as effectively as PVP). The substitution of Zwittergent 3-08 for Tween 20 is however acceptable.

### Example 6: Comparison of Method with Qiagen™ Kit

As in previous experiments either free DNA (trout) or cellular (mouse) was extracted and quantified by qPCR using the appropriate trout primers for detection of free DNA and mouse primers for the detection of cellular DNA.

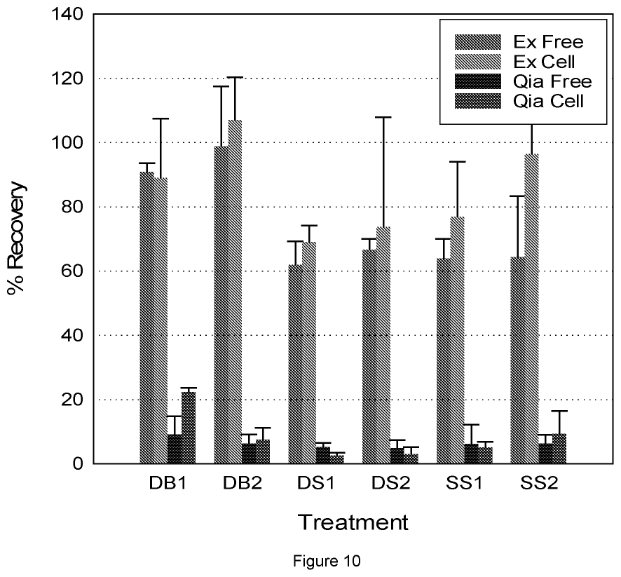

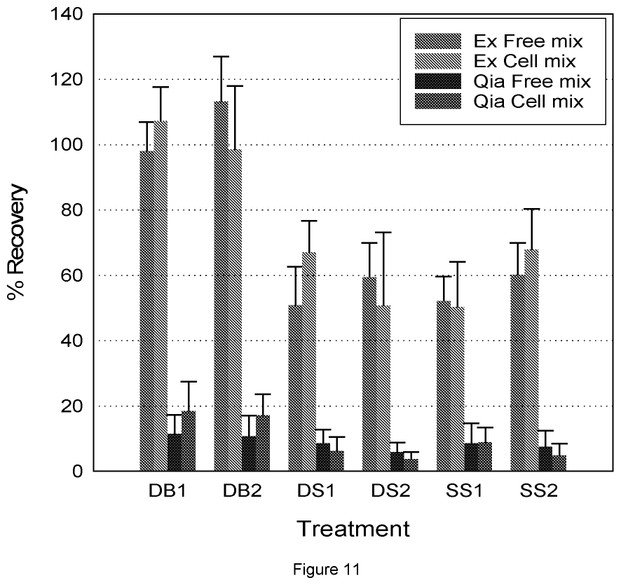

The results are shown in FIGS. 10-11.

The data shows the percentage recovery of two different amounts of the input DNA where samples denoted 2 have 50% the amount of samples denoted 1. The DNA was extracted from buffer alone (DB), swabs with the DNA directly applied to the swab (DS) and where the DNA was first applied to glass slides then swabbed (SS). Panel A shows the % recovery of free DNA and Cellular DNA applied to buffer or swabs as described above as recovered using either the Qiagen kit or extraction buffer. Panel B shows the % recovery of free DNA and Cellular DNA applied to buffer or swabs as for panel A with the exception that the cellular and free DNA was applied as a combined mixture. The data clearly shows that the extraction buffer consistently outperforms the Qiagen kit in terms of % DNA recovered in all conditions.

1. A method of eluting nucleic acid from a swab comprising cellulose, the method comprising incubating the swab in an elution solution, the elution solution comprising:
a) a polymer comprising pyrrolidone side chains;
b) a non-ionic or zwitterionic detergent;
c) a protease; and
d) a buffer at pH 5-8.5.

2. The method of claim 1, wherein the polymer is polyvinylpyrrolidone (PVP).

3. The method of claim 1, wherein the detergent is a polysorbate.

4. The method of claim 1, wherein the incubation is carried out at 50-60° C. for at least 20 minutes.

5. The method of claim 1, wherein after incubating the swab in the elution solution, the swab is centrifuged to enable extraction of the elution solution from the swab.

6. The method of claim 1, wherein the protease is proteinase K.

7. The method of claim 1, wherein the method additionally comprises heating the collected elution solution to at least 90° C. to destroy the protease activity.

8. The method of claim 1, wherein PCR is performed on the elution solution.

9. The method of claim 1, wherein before incubating the swab in the elution solution, the swab is washed with ethanol.

10. A kit for eluting nucleic acid from a swab comprising cellulose, the kit comprising:
a) a polymer comprising pyrrolidone side chains; and any one or more of the following:
b) a non-ionic or zwitterionic detergent, preferably polysorbate 20;
c) a protease, preferably proteinase K;
d) a buffer at pH 5-8.5; and
e) one or more spin baskets.

11. An elution solution comprising:
a) a polymer comprising pyrrolidone side chains; and
b) a non-ionic or zwitterionic detergent.

12. The method of claim 1, wherein the detergent is polysorbate 20.

13. The kit of claim 10, wherein the polymer comprises PVP.

14. The elution solution of claim 11, further comprising:
c) a buffer at pH 5-8.5.
